(19) United States
(12) Patent Application Publication
(10) Pub. No.: US 2009/0156688 A1
(43) Pub. Date: Jun. 18, 2009

(54) SUPPRESSANT OF TOXICITY INDUCED BY CANCER CHEMOTHERAPEUTIC AGENT AND COMPOSITION OF CANCER CHEMOTHERAPEUTIC AGENT CONTAINING THE SAME

(75) Inventors: Kwang-Kyun Park, Seoul (KR); Won-Yoon Chung, Seoul (KR); Gyoung-Ok Hong, Seoul (KR); Jae-Kwan Hwang, Gyeonggi-do (KR)

Correspondence Address: EDWARDS ANGELL PALMER & DODGE LLP, P.O. BOX 55874, BOSTON, MA, 02205, US

(73) Assignee: Biocare Co. Ltd.

(21) Appl. No.: 12/338,912

(22) Filed: Dec. 18, 2008

Related U.S. Application Data
(62) Division of application No. 10/562,412, filed on Dec. 23, 2005.

(30) Foreign Application Priority Data
Jun. 24, 2003 (KR) 10-2003-0040937

(51) Int. Cl. A61K 31/05

(52) U.S. Cl. 514/729

(57) ABSTRACT

Disclosed are a suppressant of toxicity such as hepatotoxicity and nephrotoxicity, induced by cancer chemotherapeutic agent, and a composition of cancer chemotherapeutic agent containing the suppressant. The suppressant of toxicity induced by a cancer chemotherapeutic agent contains xanthorrhizol as an active ingredient. Xanthorrhizol shows an excellent ability in suppressing ill effects generated by dosage of cancer chemotherapeutic agent, such as hepatotoxicity and nephrotoxicity.

### TECHNICAL FIELD

The present invention relates to a suppressant of toxicity induced by a cancer chemotherapeutic agent and an anti-cancer composition containing the same.

### BACKGROUND ART

Cancer is a serious disease causing about 7 million persons to die every year in the world, and it is reported that new cancer patients over 1.5 million came out in 1997 only in the United States. If taking these circumstances into consideration, cancer will be the first cause of death soon. Many methods for treating cancer such as radiotherapy, surgical therapy and gene therapy have been developed. One of the most frequently used methods is administration of a cancer chemotherapeutic agent.

Although a anti-cancer drug is a chemotherapeutic agent that selectively operates on cancer cells by the difference of sensitivity between normal cells and cancer cells, it also has problem causing toxicity to normal cells.

Cisplatin (cis-diamminedichloroplatinum [II]) is a representative platinum-based chemotherapeutic agent. This agent has been broadly used in many cancers like ovarian cancer, bladder cancer, lung cancer, cervical cancer and orchidic cancer (Rosenberg B., Cancer, 55: 2303-2315, 1985). Cisplatin is known to have anti-cancer effect through causing inter-intra strand cross-linking of DNA and making DNA additives at cancer cells. However, cisplatin causes undesirable side effects such as loss of auditory sense, neurotoxicity and nephrotoxicity when it is administered over limited dose (Mollman et al., 1998; Screnci and McKeage, 1999) and hepatotoxicity when a high dose of it is administered (Cerosimo R. J., Ann. Pharm., 27: 438-441, 1993; Cavalli F. et al., Cancer Treat. Rep., 62: 2125-2126, 1978; Pollera C. F. et al., J. Clin. Oncol., 5: 318-319, 1987).

Therefore, it has been required to develop an anti-cancer drug that has the least toxicity agent or a suppressant of toxicity induced by a cancer chemotherapeutic agent for helping a chemotherapeutic agent do adequate effects and be used safely. The co-administration of cisplatin with glutathione ester may due to effectively suppress nephrotoxicity induced by cisplatin (Babu E. et al., Mol. Cell Biochem., 144: 7-11, 1995), and a method of suppressing the toxicity of cisplatin by taking dietary antioxidant has attracted public attention (Appenroth D. et al., Arch. Toxicol., 71:677-683, 1997; Bogin E. et al., Eur. J. Clin. Chem. Clin. Biochem., 32: 843-851, 1994; and Rao M. et al., J. Biochem., 125: 383-390, 1999).

### DISCLOSURE OF INVENTION

Therefore, the technical purpose of the present invention is to solve these problems, that is, to provide a suppressant of toxicity like nephrotoxicity and hepatotoxicity induced by a cancer chemotherapeutic agent and an anti-cancer composition containing the same.

In the present invention, examples of a chemotherapeutic agent that causes toxicity include, but are not limited to, cisplatin (cis-diamminedichloroplatinum [II]), carboplatin, oxaliplatin, nedaplatin and mixture thereof.

In addition to that, the present invention provides an anti-cancer composition comprising a cancer chemotherapeutic agent and xanthorrhizol, wherein the xanthorrhizol suppresses a toxicity induced by the cancer chemotherapeutic agent.

### BRIEF DESCRIPTION OF THE DRAWINGS

These and other features, aspects and advantages of preferred embodiments of the present invention will be more fully described in the following detailed description, taken accompanying drawings. In the drawings:

FIG. 1 shows DNA-binding activities of NF-κB (A) and AP-1 (B) to evaluate the effect of xanthorrhizol on cisplatin-induced hepatotoxicity. The DNA-binding activities of NF-κB and AP-1 were evaluated by EMSA (electrophoretic mobility shift assay) using liver tissues. The filled arrow indicated each transcription factor-DNA complex of NF-κB and AP-1 and the open arrow indicated the position of the unbound oligonucleotide probe. The density of band was measured by RFLPscan software.

FIG. 2 shows mRNA expression levels of COX-2 and iNOS. The mRNA expression levels of NF-κB-dependent genes, COX-2 and iNOS, were evaluated by semiquantitative RT-PCR using specific primer sets. β-actin and GAPDH were used as control.

FIG. 3 shows results of DDRT-PCR and semiquantitative RT-PCR. (A) Two upregulated genes, S100A9 and Kin, and (B) two downregulated genes, Clpx and CP, by cisplatin were shown. The mRNA expression level of each gene was confirmed by semiquantitative RT-PCR with specific primer sets and GAPDH gene as a control.

### BEST MODES FOR CARRYING OUT THE INVENTION

Hereinafter, a suppressant of toxicity induced by a cancer chemotherapeutic agent and an anti-cancer composition comprising the same will be described in detail.

Xanthorrhizol of the present invention, which is used as an active ingredient in a suppressant of toxicity induced by a cancer chemotherapeutic agent, is a sesquiterpene compound firstly isolated from Curcuma xanthorrhiza in 1970 by German Rimpler.

This xanthorrhizol dose-dependently limits tonic contraction of rat uterine (Ponce-Monter H., et al., Phytother. Res., 13: 202-205, 1999), and has antibacterial effect against oral bacteria such as Streptococcus mutans (Hwang J. K., Fitoterapia, 71: 321-323, 2000; Hwang J. K., Planta Med., 66: 196-197, 2000). In addition to that, xanthorrhizol is known to be effective for treating or preventing cancer.

The present inventors discovered the fact that xanthorrhizol has a strong suppressing effect on toxicity like nephrotoxicity and hepatotoxicity induced by a cancer chemotherapeutic agent, following many works to develop those compounds.

Xanthorrhizol having below formula I can be extracted from Curcuma xanthorrhiza Roxb. (Zingiberaceae), which has been a medicinal plant in Indonesia. Several extracting methods like organic solvent extraction, supercritical fluid extraction, microwave extraction and ultrasonic extraction can be used, as shown in South Korea patent publication No. 2000-73295 and PCT patent No. WO88/05304.

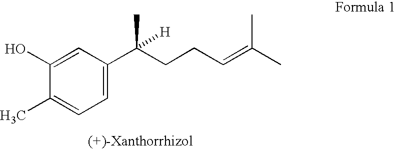

As said above, xanthorrhizol shows distinguished ability to suppress side effects like nephrotoxicity and hepatotoxicity induced by administration of a cancer chemotherapeutic agent. Examples of the chemotherapeutic agent that its side effects can be suppressed include, but are not limited to, platinum-based anticancer drug, cylcophosphamide, bleomycin and doxorubicin. In particular, the suppression of hepatotoxicity and nephrotoxicity induced by platinum-based anticancer drug like cisplatin (cis-diamminedichloroplatinum [II]), carboplatin, oxaliplatin and nedaplatin is more efficacious. The xanthorrhizol's effect on cancer chemotherapeutic agent is thought to be because xanthorrhizol suppresses operation of reactive oxygen species made by a chemotherapeutic agent.

Methods of evaluating the suppressing effects of xanthorrhizol on nephrotoxicity and hepatotoxicity induced by cisplatin, representative platinum-based chemotherapeutic agent, are followings.

Cisplatin was intraperitoneally injected in mice. After some time passed, body weights of the mice were checked and induction of toxicity was confirmed. Blood was gotten from the heart of etherized mouse, and biochemical markers related to the induction of hepatotoxicity and nephrotoxicity were evaluated, and the kidney and spleen were separated and weighed for comparison.

Determining activities of some enzymes in serum can show much information for diagnosis of many diseases. Aminotransferase is present at a high level in liver and little detected in blood. However, hepatotoxicity increases the level of aminotransferase in blood. Denatured liver, treated with cisplatin, releases GPT (Glutamate-Pyruvate Transaminase) and GOT (Glutamate-Oxaloacetate Transaminase) from injured liver cells into blood. Group that had the oral pretreatment of xanthorrhizol before cisplatin was intraperitoneally injected showed significantly decreased concentration of GPT and GOT in blood in comparison with group administrated with only cisplatin.

Changes of specific gravity of kidney were also measured to evaluate suppressing effects of xanthorrhizol on nephrotoxicity induced by cisplatin. Group administered high dose of cisplatin showed increased specific gravity of kidney in comparison with control group without being administered cisplatin. However, group that had oral pretreatment of xanthorrhizol for some days before cisplatin injection showed almost changeless specific gravity of kidney.

In addition, nephrotoxicity induced by cisplatin increases reactive oxygen species, and efficiency of filtration and excretion of kidney decreases, and changes of body weight also happen. The levels of urea nitrogen and creatinine in the blood increase because of lowering of filtration function. Group that administered xanthorrhizol before cisplatin injection showed to significantly decrease the blood level of urea nitrogen in comparison with group that took with only cisplatin.

Administration of platinum-based chemotherapeutic agent like cisplatin is known to activate or suppress transcription factors such as NF-κB (nuclear factor-kappa B) and AP-1 (activator protein-1), and the activation of these transcription factors, especially NF-κB, cause activation of NF-κB-dependent genes such as COX-2 (cyclooxygenase-2) and iNOS (inducible nitric oxide synthase), which are well-known pro-inflammatory genes that are associated with inflammation and toxicity (Nanji, A. A., et al., 2003. Am. J. Physiol.: Gastronintest. Liver Physiol. 284, G321-27; Reilly, T. P., et al., Chem. Res. Toxicol. 14, 1620-1628; and Gardner, C. R., et al., Hepatology 27, 748-754).

The physiological function of curcumin, which is used as a comparative example in the present invention, is known to be closely associated with its ability to inhibit the activated transcription factors such as NF-κB, especially (Han, S. S., et al., 2002. J. Biochem. Mol. Biol. 35, 337-342 and Nanji, A. A., et al., 2003. Am. J. Physiol.: Gastronintest. Liver Physiol. 284, G321-27).

We confirmed activation of NF-κB by cisplatin in the present invention. The elevated mRNA expression of COX-2 and iNOS genes by cisplatin was also confirmed in the present invention. The pretreatment of either xanthorrhizol or curcumin prior to administration of cisplatin suppressed mRNA expression of these genes. Xanthorrhizol suppressed both mRNA expression of COX-2 and iNOS genes induced by cisplatin but curcumin suppressed only expression of COX-2 gene. This fact means xanthorrhizol is efficacious on treatment of hepatotoxicity induced by cisplatin.

In addition to that, to identify the differentially expressed genes related in the prophylactic effect of xanthorrhizol on cisplatin-induced hepatotoxicity, DDRT-PCR (differential display reverse transciption-PCR) technique was performed, and seven upregulated genes and five downregulated genes by cisplatin were identified.

Among these genes, the upregulated S100 calcium binding protein A9 (S100A9) mRNA by cisplatin could be explained by the fact that cisplatin decreases DNA-binding activity of AP-1 that negatively function S100A9 mRNA expression (Gebhardt, C., et al., 2002. Oncogene 21, 4266-4276). This S100A9 has been suggested to affect alteration of the cytoskeleton and cell shape, signal transduction (Kerkhoff, C., et al., J. Biol. Chem. 274, 32672-32679), and modulation of intracellular calcium (Schafer, B. W., et al., Trends Biochem. Sci. 21, 134-140). The aberrant expression of S100A9 mRNA by cisplatin is ultimately related to the impairment of Ca²⁺ regulation, and cisplatin-induced hepatotoxicity could be highly linked with the perturbation of Ca²⁺ homeostasis. Administration of xanthorrhizol, in contrast with curcumin, is thought to abrogate the cisplatin-induced inhibition of AP-1, which decreases the mRNA expression of S100A9 and obtains the desired effect on hepatotoxicity induced by cisplatin after all.

Antigenic determinant of rec-A protein (Kin) is a nuclear protein, which presents cross-immunoreactivity with the bacterial RecA protein and efficiently binds to curved DNA (Tissier, A., et al., Biochimie 77, 854-860). This genomic interaction could be implied in DNA repair and illegitimate recombinant in eukaryotic cells. The expression of Kin mRNA by cisplatin increased the level of the Kin protein (Angulo, J. F., et al., Mutat. Res., 217, 123-134). Because enhancement of DNA repair activity in rat-liver cells exposed to cisplatin has been reported, increased Kin mRNA in cisplatin-treated mice could reflect its ability to repair DNA damage induced by cisplatin in liver. As consistent with the effect of xanthorrhizol on the expression of iNOS, the upregulated mRNA expression levels of Kin by cisplatin were dramatically reduced by the pretreatment of xanthorrhizol. This means xanthorrhizol has a suppressing effect on toxicity induced by cisplatin.

Administration of cisplatin can also produce marked changes in mitochondria. Exposure to cisplatin resulted in the inhibition of complex I and complex II activities of respiratory chain in mouse liver (Rosen, M., et al., Int. J. Exp. Pathol. 73, 61-74) and the loss of mitochondrial membrane potential (Kruidering, M., et al., Exp. Nephrol. 2, 334-344) that consequently affects the whole function of mitochondria.

Mitochondrial dysfunction in hepatotoxicity could be also explained with another aberrantly expressed gene, murine ClpX (caseinolytic proteinase X). ClpX protein displays intrinsic ATPase activity and acts as a tissue-specific mammalian mitochondrial chaperone that may play a role in mitochondrial protein homeostasis (Santagata, S., et al., J. Biol. Chem. 274, 16311-16319). Its decreased expression could result in the instability of mitochondria, but the pretreatment of xanthorrhizol maintained the ClpX mRNA expression in the same level as without administrating with cisplatin. This also means administration of xanthorrhizol attenuates the toxicity induced by administration of cisplatin.

Ceruloplasmin (CP) is a serum α 2-glycoprotein containing greater than 95% of the total copper found in the plasma of vertebrate species (Takahashi, N., et al., Proc. Natl. Acad. Sci. U.S.A. 81, 390-394). CP acts as a protective antioxidant in plasma by tightly binding the plasma copper and inhibits iron-dependent lipid peroxidation and hydroxyl radical formation. Interestingly, administration of cisplatin induced a fall in levels of plasma antioxidant proteins including CP. This may reflect a failure of the antioxidant defense mechanism against oxidative damage induced by commonly used anticancer drug (Weijl, N. I., et al., Ann. Oncol. 9, 1331-1337). We confirmed that the pretreatment of xanthorrhizol slightly recovered the mRNA expression of CP, which means xanthorrhizol is used as a suppressant of toxicity induced by a chemotherapeutic agent. Administration of curcumin as a comparative example did not show that effect as described above.

On the basis of above facts and results, xanthorrhizol is thought to have a good effect on suppressing undesirable side effects like nephrotoxicity and hepatotoxicity induced by a cancer chemotherapeutic agent, and the efficacy of xanthorrhizol is thought to be better than that of curcumin that can be used as a suppressant of toxicity.

Xanthorrhizol can be administered through various routes. Routes of administration include, but are not limited to, oral, topical, subcutaneous, transdermal, subdermal, intra-muscular, intra-peritoneal, intra-articular, intra-arterial, intra-venous, intra-dermal, intra-lesional, intra-ocular, intra-pulmonary and intra-spinal. It can be formulated into solution, suspension, emulsion, tablets, capsules and sustained release system.

The dose of xanthorrhizol may be adjusted depending on dose and species of a cancer chemotherapeutic agent, age of patient, sex of patient and so on. Xanthorrhizol can be administered alone before or after administration of a cancer chemotherapeutic agent and can also be administered as an anti-cancer composition with a chemotherapeutic agent.

Depending on the condition of patient, the dose of a platinum-based chemotherapeutic agent, the period of administration and some circumstances, the amount of xanthorrhizol administered per dose may be adjusted. Preferably, the amount of administered xanthorrhizol is from about 0.01 to 10 times by total administrated weight of cisplatin, more preferably from about 0.1 to 5 times weight by total weight of administered cisplatin.

The present invention provides an anti-cancer composition comprising a cancer chemotherapeutic agent and xanthorrhizol, wherein the xanthorrhizol suppresses toxicity induced by the cancer chemotherapeutic agent. The amount of xanthorrhizol in the composition is preferably from about 0.01 to 10 times, more preferably from about 0.1 to 5 times by weight of the cancer chemotherapeutic agent.

A suppressant of toxicity induced by a cancer chemotherapeutic agent comprising xanthorrhizol and an anti-cancer composition comprising the same can further comprise pharmaceutically acceptable additives and diluents. Additives and diluents include, but are not limited to, common fillers, binders, lubricants, wetting agents, suspending agents, solvents, dispensing agents, controlled releasing agents, flavors, colorants and coating agents.

Hereinafter, the present invention is described in considerable detail to help those skilled in the art understand the present invention. However, the following examples are offered by way of illustration and are not intended to limit the scope of the invention. It is apparent that various changes may be made without departing from the spirit and scope of the invention or sacrificing all of its material advantages.

The results of below examples are shown as average value±SE. Statistical analysis was performed by Student t-test, and the level of significance was considered at P value<0.05.

### Experimental Example 1

### Design of Animal Model

Suppressing effects of xanthorrhizol and curcumin on cisplatin-induced hepatotoxicity and nephrotoxicity were compared. Each example group consisted of 10 ICR mice (5 weeks, male). Mice were dosed with xanthorrhizol (100 or 200 mg/kg per day, in corn oil) or curcumin (200 mg/kg per day, in PBS buffer) orally for four consecutive days. Only the corn oil was dosed orally as a negative control. Three hours after the last treatment of xanthorrhizol or curcumin, 45 mg/kg of cisplatin (in PBS buffer) was intraperitoneally injected, and the PBS buffer was injected as a negative control. Mice were weighed 16 h after the injection, killed under ether anesthesia and blood and liver samples were collected. The kidney and spleen were separated and weighed, respectively. Administration route and dosage according to examples and comparative examples are shown in Table 1.

TABLE 1

| | Route, Dosage | |
|---|---|---|
| | Oral | intraperitoneal |
| | administration, | injection, |
| Group | 4 days | 16 hrs |
| Control | Corn oil | Buffer solution |
| Comparative example 1 | Corn oil | Cisplatin 45 mg/kg |
| (Cisplatin administration) | | |
| Comparative example 2 | Curcumin | Cisplatin 45 mg/kg |
| (Curcumin administration) | 200 mg/kg | |
| Example 1 | Xanthorrhizol | Cisplatin 45 mg/kg |
| (Xanthorrhizol | 100 mg/kg | |
| administration 1) | | |
| Example 2 | Xanthorrhizol | Cisplatin 45 mg/kg |
| (Xanthorrhizol | 200 mg/kg | |
| administration 2) | | |

### Experimental Example 2

### Determination of Serum Biochemical Parameters

Blood samples obtained from heart were kept at room temperature for 2 h, centrifuged at 300 rpm for 10 minutes to obtain sera, and stored at low temperature for analyzing proteins. GPT (Glutamate-Pyruvate Transaminase), GOT (Glutamate-Oxaloacetate Transaminase), blood urea nitrogen (BUN), and creatinine from the serum were measured. The results are shown in Table 2.

Quantitative Method of GPT (Glutamate-Pyruvate Transaminase) and GOT (Glutamate-Oxaloacetate Transaminase)

Activities of GPT and GOT were determined by the method of Reitman and Frankel (1957). The principle is following: α-ketoglutaric acid+alanine→glutamate+pyruvate. Pyruvate formed by GPT enzyme in said reaction is reacted with 2,4-dinitrophenylhydrazine and the intensity of color formed by the resulting reaction is related to enzymatic activity. The absorbance was determined at 505 nm. The reagents for determining GPT and GOT were purchased from Sigma chemical Co. (St. Louis, U.S.A.). Because hemolyzed serum contains a high level of GOT and GPT compared to normal serum, only unhemolyzed serum should be used as much as possible. The separated serum samples were stored at 4° C. and used within 5 days because the activity of GPT and GOT might decrease after 5 days at low temperature.

1 ml alanine-α-KG substrate was added to test tube and was pre-warmed at 37° C. for 2-3 minutes. 0.2 ml serum samples was added to each tube and incubated at 37° C. for 30 minutes in water bath. After that, 1 ml 2,4-dinitrophenylhydrazine was added and incubated at room temperature for 20 minutes. 10 ml 0.4N NaOH solution was added to each tube and was mixed well. After that, the GPT absorbance of each tube was read comparing with distilled water. In case of GOT determination, 1 ml aspartate-α-KG substrate was pre-warmed at 37° C. for 2-3 minutes, and 0.2 ml serum samples were added to each tube and incubated at 37° C. for 60 minutes, and the absorbance was read.

Quantitative Method of Blood Urea Nitrogen

The content of urea nitrogen was assayed by the method of Faweett et al. (1957). Through a kit for assaying blood urea nitrogen, Ammonia (NH₃) formed by hydrolysis of urea was measured by the absorbance at 570 nm.

0.5 ml urease solution was added to test tube and mixed with 10 μl serum sample, and incubated in water bath at 37° C. for 5-10 minutes. 1 ml phenol nitroprusside solution and 1 ml alkaline hypochlorite solution were added to the tube and mixed gently, and 5 ml distilled water was added. After that, the absorbance of the reaction was measured comparing with distilled water as a control. The reagents for determining urea nitrogen were purchased from Sigma chemical Co. (St. Louis, U.S.A.) and standard calibration curve was used.

Quantitative Method of Creatinine

The content of creatinine was determined by the method of Jaffe et al. (1886), which a yellow color caused by treating creatinine metabolites with alkaline picrate was measured in the absorbance at 500 nm. Standard calibration curve was used.

3 ml alkaline picrate solution and 0.3 ml serum sample were mixed well at 37° C. for 20 minutes and the absorbance (A1) was measured. Distilled water was used for a negative control. 0.1 ml acid solution (mixture of sulfuric acid and acetic acid) was added to each tube and incubated in water bath at 37° C. for 5 minutes. After that, the absorbance (A2) was read with distilled water as a negative control. Absorbance of the sample for determining the content of creatinine is absorbance (A1) minus absorbance (A2) according to standard calibration curve. The reagents for determining creatinine were purchased from Sigma chemical Co. (St. Louis, U.S.A.)

TABLE 2

| | K.W/B.W | S.W/B.W | GPT | GOT | BUN | Creatinine |
|---|---|---|---|---|---|---|
| Group | *1000% | *1000% | (U/liter) | (U/liter) | (mg/dL) | (mg/dL) |
| Control | 15.2 ± 1.3 | 3.1 ± 0.4 | 56.4 ± 11.2 | 157.6 ± 38.8 | 20.3 ± 3.1 | 0.31 ± 0.5 |
| Comparative | 19.2 ± 1.6 | 2.1 ± 0.4 | 185.8 ± 86.3 | 517.1 ± 99.1 | 144.4 ± 20.6 | 2.8 ± 0.7 |
| example 1 | | | | | | |
| (Cisplatin) | | | | | | |
| Comparative | 17.2 ± 1.7** | 2.0 ± 0.1 | 158.4 ± 84.3 | 381.6 ± 144.7* | 138.6 ± 46.2 | 2.2 ± 1.4 |
| example 2 | | | | | | |
| (Curcumin) | | | | | | |
| Example 1 | 17.5 ± 2.9 | 2.1 ± 0.4 | 134.2 ± 58.5 | 296.5 ± 74.5*** | 145.2 ± 23.5 | 2.0 ± 1.1 |
| (Xanthorrhizol, | | | | | | |
| 100 mg/kg) | | | | | | |
| Example 2 | 14.6 ± 0.9*** | 2.0 ± 0.3 | 106.0 ± 28.3** | 201.4 ± 50.3*** | 50.9 ± 16.7*** | 0.8 ± 0.5*** |
| (Xanthorrhizol, | | | | | | |
| 200 mg/kg) | | | | | | |

*P < 0.05. **P < 0.01, ***P < 0.0001

In table 2, K.W/B.W means kidney weight/body weight, S.W/B.W means spleen weight/body weight, and BUN means Blood Urea nitrogen.

As shown in table 2, the group that had the oral pretreatment of xanthorrhizol (200 mg/kg) for 4 days before cisplatin was injected intraperitoneally showed significantly reduced GPT level in comparison with the group administrated by only cisplatin, and the pretreatment of xanthorrhizol was more efficacious than the pretreatment of curcumin (200 mg/kg). The group administered high dose of cisplatin also showed the increased specific gravity of kidney compared to the group without not being administered with cisplatin, but the oral pretreatment of curcumin and xanthorrhizol for 4 days before cisplatin injection decreased specific gravity of kidney at the level similar to that of the control group and xanthorrhizol was more efficacious than curcumin.

The group administered with xanthorrhizol (200 mg/kg) for 4 days before cisplatin administration also showed more significantly decreased level of urea nitrogen in blood compared to that of the group administered only cisplatin. The group administered with xanthorrhizol (200 mg/kg) before cisplatin administration showed significantly decreased level of creatinine in blood, while the group having a nephrotoxicity induced by administrating cisplatin showed a increased level of creatinine in blood.

### Experimental Example 3

### Evaluation of Xanthorrhizol's Effects on NF-κB and AP-1

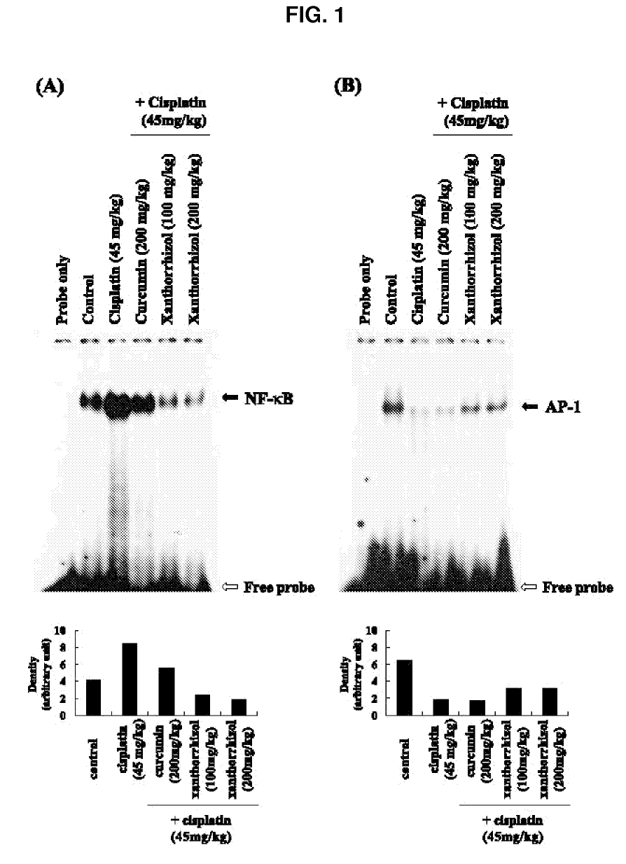

EMSA (electrophoretic mobility shift assay) was performed to evaluate the xanthorrhizol's effects on NF-κB and AP-1. Liver tissues of example 1, example 2, comparative example 1 and comparative example 2, which were made at the above experimental example 1, were powdered under liquid nitrogen. After that, powdered liver tissues were homogenized in 500 μl of cool hypotonic buffer [10 mM HEPES (pH 7.8), 10 mM KCl, 1.5 mM MgCl₂, 0.5 mM DTT, 0.2 mM PMSF]. To the homogenates was added 125 μl of 10% NP-40 solution, and the mixture was then centrifuged at 12,000×g for 1 min. Pellets were washed once with 100 μl of the above buffer and 12.5 μl of 10% NP-40, centrifuged, resuspended in 50 μl of 20 mM cool HEPES buffer (pH 7.8) containing 420 mM NaCl, 1.5 mM MgCl₂, 0.2 mM EDTA, 0.5 mM DTT, 0.2 mM PMSF, and 20% glycerol, and centrifuged at 12000×g for 5 min at 4° C. The supernatant containing nuclear proteins was collected and assayed protein concentration and stored at −70° C. Either NF-κB oligonucleotide probe (5′-AGT TGA GGG GAC TTT CCC AGG C-3′; Promega, Wisconsin) or AP-1 (c-Jun) oligonucleotide probe (5′-CGC TTG ATG AGT CAG CCG GAA-3′; Promega, Wisconsin) was labeled with [γ-³²P]ATP by T4 polynucleotide kinase and purified on a Nich column (Pharmacia, Uppsala, Sweden). The binding reaction was carried out in 25 μl of the mixture containing 5 μl of incubation buffer [10 mM Tris-HCl (pH 7.5), 100 mM NaCl, 1 mM DTT, 1 mM EDTA, 4% (v/v) glycerol, and 0.1 mg/ml sonicated salmon sperm DNA], 10 μg of nuclear extracts, and 100,000 cpm of the labeled probe. After 50 min of incubation at room temperature, the samples and comparative samples mixed with 3 μl of loading buffer (250 mM Tris-HCl (pH7.5), 0.2% bromophenol blue, 40% glycerol) were electrophoresed through a 6% non-denaturing polyacrylamide gel at 150 V for 2 hrs. Finally, the gel was dried and exposed to X-ray film. The results are shown in FIG. 1.

As shown in FIG. 1, the treatment of cisplatin showed the increased DNA-binding activity of NF-κB, but in contrast, the decreased DNA-binding activity of AP-1. However, the pretreatment of xanthorrhizol and curcumin suppressed the binding activity of NF-κB induced by cisplatin. If the same dosage is administrated Xanthorrhizol's effect was much stronger than those shown in curcumin-treated groups. The pretreatment of xanthorrhizol recovered the suppressed binding activity of AP-1 by cisplatin by about 50%, but the pretreatment of curcumin did not change the suppressed binding activity of AP-1.

### Experimental Example 4

### Evaluation of Xanthorrhizol's Effect at Gene Level

Isolation of Total RNAs and DNase I Digestion

Liver tissues of example 1, example 2, comparative example 1 and comparative example 2, which were made at the above experimental example 1, were powdered under liquid nitrogen. After that, powdered liver tissues were homogenized using TRIzol™ reagent (Life technologies, Austria). The homogenized samples were incubated for 5 min at room temperature to permit the complete dissociation of nucleoprotein complexes. After the addition of 0.2 volume of chloroform, samples were shaken vigorously for 15 seconds, incubated for 2-3 min, and centrifuged at 12000×g for 15 min at 4° C. Total RNA in the upper aqueous phase was precipitated by mixing with an equal volume of isopropanol. The mixtures were incubated for 10 min at 4° C. and centrifuged at 12000×g for 10 min at 4° C. Total RNA pellet was washed with 75% ethanol, dried, and dissolved in RNase free water. To prevent the contamination with chromosomal DNA, total RNA samples were incubated with 10 units of DNase I (GenHunter Corp., Nashville, USA) at 37° C. for 30 min, and then DNA-free RNA was isolated using TRIzol reagent. The concentration and purity of total RNA and DNase-I-treated total RNA were calculated with absorbance at 260 and 280 mm.

DDRT-PCR

DDRT-PCR (differential display reverse transciption-polymerase chain reaction) was performed using the RNAimage kit (GenHunter Corp., Nashville, USA). The DNase I-treated total RNA pools (200 ng per each group) were carry out reverse transcription reaction in reverse transcriptase buffer (25 mM Tris-HCl, pH 8.3, 37.6 mM KCl, 1.5 mg MgCl₂, and 5 mM DTT) with 5 unit/μl of MMLV-reverse transcriptase, 20 μM dNTP mix, and 0.2 μM of guanosine-anchored oligo(dT) primer (HT₁₁-G). The RT mixture was diluted at 1:10 and used for PCR. Subsequent PCR (20 μl) was performed in PCR buffer (10 mM Tris-HCl, pH 8.4, 50 mM KCl, 1.5 mM MgCl₂ and 0.001% gelatin) containing 2 μM dNTP, 0.2 μM of HT₁₁-G, 0.2 μM of a primer (from H-AP1 to H-AP10), 0.2 μl of α-[³³P]dATP (2000 Ci/mmol), and 0.05 unit/μl of AmpliTaq DNA polymerase (Perkin-Elmer). The thermocycler (GeneAmp PCR System 9700, Perkin-Elmer) was programmed as follows: 40 cycles at 94° C. for 30 s, 40° C. for 2 min, and 72° C. for 30 s, and terminated with a final elongation at 72° C. for 5 min. ³³P-labeled PCR products were separated on 6% denaturing polyacrylamide gel for 3.5 h at 60 W constant powers. The blotted gel on a piece of 3 M paper was dried under vacuum at 80° C. for 1 h. The autoradiogram oriented with the dried gel was exposed and developed.

Cloning and DNA Sequencing

The interesting cDNA fragments were cut from the dried gel, eluted by boiling in water, and reamplified by PCR with the same set of primers at same PCR conditions used in DD-PCR. The reamplified PCR products were cloned in PCR-TRAP vector using PCR-TRAP cloning system (GenHunter) according to the manufacturer's instructions. DNA sequencing for plasmids containing DNA inserts was performed at Takara Korea Biomedical Inc. (Suwon, Korea), and the sequence alignment was performed in GenBank of National Center for Biotechnology Information (NCBI) using standard nucleotide-nucleotide BLAST (blastn) program and all EMBL libraries using Fasta3 program.

Primer Design and RT-PCR

Semiquantitative RT-PCR was performed to confirm the results from DDRT-PCR. To set the most suitable PCR amplification conditions, primers for interesting gene were determined by an on-line primer design program (Rozen and Skaletsky, 2000). Used primer sets in the present invention were shown in Table 3.

TABLE 3

| | | |
|---|---|---|
| | | Product Size |
| Target gene | Sequence | (bp) |

| | | | |
|---|---|---|---|
| COX-2 | forward | 5′-GGA GAG ACT ATC AAG ATA GTG | 861 |
| | (SEQ ID NO: 1) | ATC-3′ | |
| | reverse | 5′-ATG GTC AGT AGA CTT TTA CAG | |
| | (SEQ ID NO: 2) | CTC-3′ | |
| iNOS | forward | 5′-AAG TTC AGC AAC AAC CCC AC-3′ | 560 |
| | (SEQ ID NO: 3) | | |
| | reverse | 5′-TCC TGA ACG TAG ACC TTG GG-3′ | |
| | (SEQ ID NO: 4) | | |
| S100A9 | forward | 5′-AGG ACC TGG ACA CAA ACC AG-3′ | 230 |
| | (SEQ ID NO: 5) | | |
| | reverse | 5′-TCA TTT CCC AGA ACA AAG GC-3′ | |
| | (SEQ ID NO: 6) | | |
| Kin | forward | 5′-GAC AAC TGT TGC TGG CTT CA-3 | 527 |
| | (SEQ ID NO: 7) | | |
| | reverse | 5′-TGG TCC CAA AGA GCT TGA CT-3′ | |
| | (SEQ ID NO: 8) | | |
| ClpX | forward | 5′-GCG CAG AGC TCC TCT TAG AA-3′ | 505 |
| | (SEQ ID NO: 9) | | |
| | reverse | 5′-CTT CTC AGC CTC TGC TTG CT-3′ | |
| | (SEQ ID NO: 10) | | |
| Cp | forward | 5′-TGC TCT GAA CCC GAG AAA GT-3′ | 449 |
| | (SEQ ID NO: 11) | | |
| | reverse | 5′-CCA GAG GGA GCA TAA TTC CA-3′ | |
| | (SEQ ID NO: 12) | | |
| β-actin | forward | 5′-TAC AAT GAG CTG CGT GTG GC-3′ | 365 |
| | (SEQ ID NO: 13) | | |
| | reverse | 5′-ATG TCA CGC ACG ATT TCC C-3′ | |
| | (SEQ ID NO: 14) | | |
| GAPDH | forward | 5′-CTG CAC CAC CAA CTG CTT AG-3′ | 603 |
| | (SEQ ID NO: 15) | | |
| | reverse | 5′-GCC TCT CTT GCT CAG TGT CC-3′ | |
| | (SEQ ID NO: 16) | | |

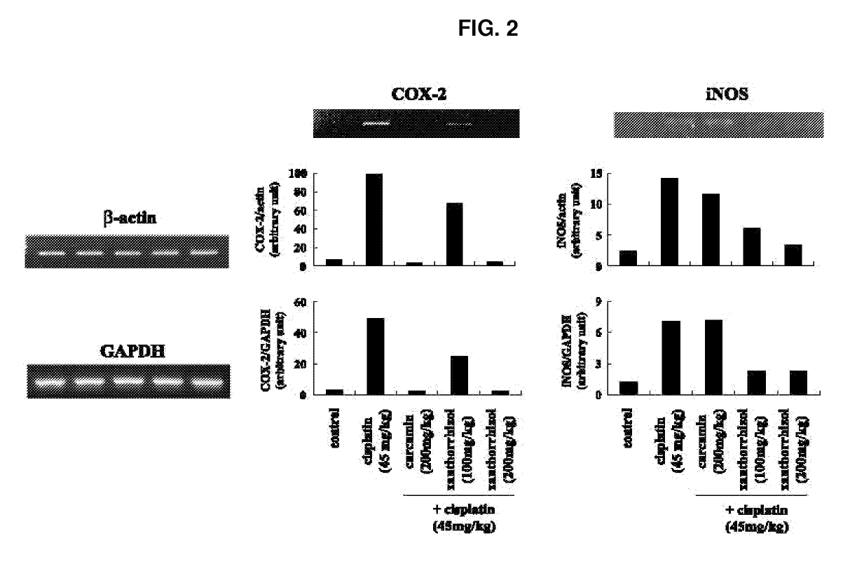

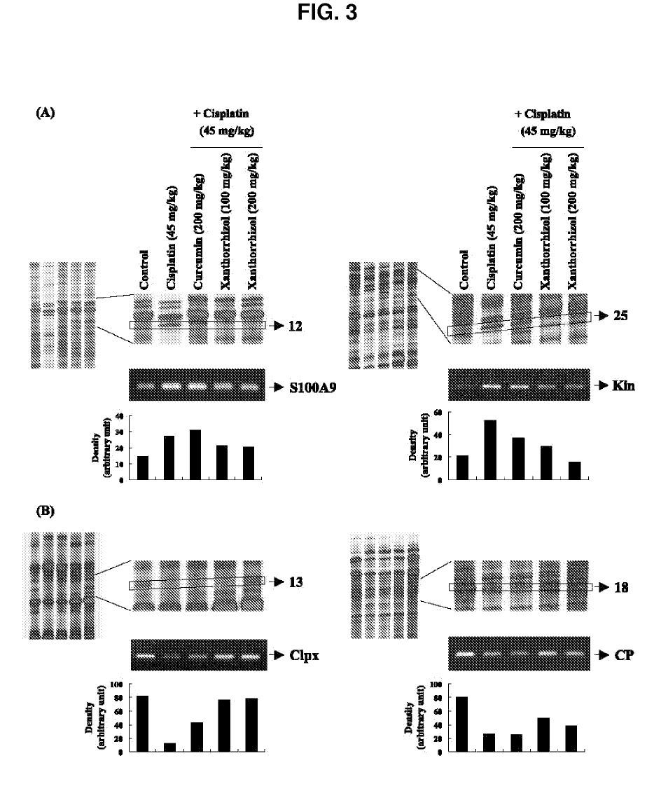

First-strand cDNA was synthesized with 1 μg of total RNAs and 1 μM of oligo-dT₁₅ primer using Omniscript Reverse Transcriptase (Qiagen, California). Using Taq PCR Master Mix kit (Qiagen), subsequent PCR was performed with 0.5 μl of first-strand cDNA and 20 pmol of primers (See Table 1). The PCR reaction consisted of initial denaturation at 94° C. for 3 min, three-step cycling (30 cycles) with denaturation at 94° C. for 40 s, annealing at 53° C. for 40 s, and elongation at 72° C. for 1 min, and final elongation at 72° C. for 10 min. The amplified PCR products were loaded into 1.2% agarose gel. After ethidium bromide staining, the gel was illuminated on the UV transilluminator and the photography was made using Polaroid DS-34 Instant Camera system (Kodak, USA). The results are shown in FIG. 2 and FIG. 3, respectively.

As shown in FIG. 2, the mRNA expression levels of NF-κB-dependent genes, COX-2 and iNOS, were evaluated by semiquantitative RT-PCR. Two kinds of house keeping genes, β-actin and GAPDH, were used to normalize each mRNA expression. As shown in FIG. 2, the two genes were highly induced by the administration of cisplatin, but the pretreatment of either xanthorrhizol or curcumin at same dose (200 mg/kg) returned the cisplatin-induced COX-2 mRNA expression to initial level. Induction of iNOS mRNA expression by cisplatin was strongly suppressed by the pretreatment of xanthorrhizol, but induction of iNOS mRNA expression by cisplatin was not suppressed by the pretreatment of curcumin.

As shown in FIG. 3, to identify the differentially expressed genes related in the protective effect of xanthorrhizol on cisplatin-induced hepatotoxicity, DDRT-PCR was performed. Using 10 sets of primer combination, 7 upregulated genes (Table 4) and 5 downregulated genes (Table 5) by cisplatin were identified. Using semiquantitative RT-PCR, it was confirmed that the mRNA expression levels of two upregulated genes (S100A9 and kin) and two downregulated genes (ClpX and CP) by cisplatin were reversed respectively by the pretreatment of xanthorrhizol. It shows the effect of xanthorrhizol is stronger than the effect of curcumin comparing with curcumin.

TABLE 4

| Clone | | | Homology |
|---|---|---|---|
| no. | Accession no. | Description | (%) |
| 4 | AK027904 | Mus musculus adult male kidney cDNA, RIKEN full-length | 98 |
| | | enriched library, clone: 0610005B19, product: hemoglobin, | |
| | | beta adult major chain, full insert sequence | |
| 6 | NM_010887 | Mus musculus NADH dehydrogenase (ubiquinone) Fe—S | 100 |
| | | protein 4 (Ndufs4), Mrna | |
| 7 | AV37808 | Mouse EST sequence, N/D | 93 |
| 8 | AK078309 | Mus musculus adult male olfactory brain cDNA, RIKEN | 99 |
| | | full-length enriched library, clone: 6430590N12, product: | |
| | | hypothetical protein, full insert sequence | |
| 12 | BC027635 | Mus musculus S100 calcium binding protein A9 (S100A9; | 100 |
| | | calgranulin B), mRNA, complete cds | |
| 23 | BC004015 | Mus musculus, clone MGC: 7593, IMAGE: 3493893, | 96 |
| | | mRNA, complete cds | |
| 25 | BC028860 | Mus musculus antigenic determinant of rec-A protein (Kin), | 100 |
| | | mRNA, complete cds | |

TABLE 5

| Clone | | | Homology |
|---|---|---|---|
| no. | Accession no. | Description | (%) |
| 13 | XM_193096.1 | Mus musculus caseinolytic protease X (E. coli) (ClpX), | 98 |
| | | mRNA | |
| 15 | AK002442 | Mus musculus adult male kidney cDNA, RIKEN full-length | 100 |
| | | enriched library, clone: 0610010A23, product: similar to | |
| | | CGI-90 protein [Homo sapiens], full insert sequence | |
| 18 | NM_007752 | Mus musculus ceruloplasmin (Cp), mRNA | 99 |
| 20 | BC025868 | Mus musculus transformed mouse 3T3 cell double minute 4, | 100 |
| | | mRNA (cDNA clone IMAGE: 5025694), partial cds | |
| 26 | AK035342 | Mus musculus adult male urinary bladder cDNA, RIKEN | 100 |
| | | full-length enriched library, clone: 9530020C10, product: | |
| | | unknown EST, full insert sequence | |

### INDUSTRIAL APPLICABILITY

As described above, xanthorrhizol is useful as a suppressant of toxicity induced by a cancer chemotherapeutic agent because xanthorrhizol shows an excellently suppressing effect on the undesirable side effects like nephrotoxicity and hepatotoxicity induced by the chemotherapeutic agent. An anti-cancer composition comprising a cancer chemotherapeutic agent and xanthorrhizol can also minimize the side effects, while the composition has the efficacy of the cancer chemotherapeutic agent.

What is claimed is:

1. A method of suppressing toxicity induced by a cancer chemotherapeutic agent, comprising administering to a subject in need thereof a therapeutically effective amount of xanthorrhizol.

2. The method of according to claim 1, wherein the toxicity is hepatotoxicity or nephrotoxicity.

3. The method according to claim 1, wherein the cancer chemotherapeutic agent is a platinum-based anti-cancer drug selected from the group consisting of cisplatin (cis-diamminedichloroplatinum [II]), carboplatin, oxaliplatin, nedaplatin and mixture thereof.

4. The method according to claim 2, wherein the cancer chemotherapeutic agent is a platinum-based anti-cancer drug selected from the group consisting of cisplatin (cis-diamminedichloroplatinum [II]), carboplatin, oxaliplatin, nedaplatin and mixture thereof.

5. The method according to claim 1, wherein the amount of xanthorrhizol is from 0.01 to 10 times by weight of the cancer chemotherapeutic agent.


SEQUENCE LISTING (16 sequences)

SEQ ID NO: 1 - DNA, 24 bp, Artificial Sequence
  forward primer for analyzing COX-2 gene expression
ggagagactatcaagatagtgatc

SEQ ID NO: 2 - DNA, 24 bp, Artificial Sequence
  reverse primer for analyzing COX-2 gene expression
atggtcagtagacttttacagctc

SEQ ID NO: 3 - DNA, 20 bp, Artificial Sequence
  forward primer for analyzing iNOS gene expression
aagttcagcaacaaccccac

SEQ ID NO: 4 - DNA, 20 bp, Artificial Sequence
  reverse primer for analyzing iNOS gene expression
tcctgaacgtagaccttggg

SEQ ID NO: 5 - DNA, 20 bp, Artificial Sequence
  forward primer for analyzing S100A9 gene expression
aggacctggacacaaaccag

SEQ ID NO: 6 - DNA, 20 bp, Artificial Sequence
  reverse primer for analyzing S100A9 gene expression
tcatttcccagaacaaaggc

SEQ ID NO: 7 - DNA, 20 bp, Artificial Sequence
  forward primer for analyzing Kin gene expression
gacaactgttgctggcttca

SEQ ID NO: 8 - DNA, 20 bp, Artificial Sequence
  reverse primer for analyzing Kin gene expression
tggtcccaaagagcttgact

SEQ ID NO: 9 - DNA, 20 bp, Artificial Sequence
  forward primer for analyzing ClpX gene expression
gcgcagagctcctcttagaa

SEQ ID NO: 10 - DNA, 20 bp, Artificial Sequence
  reverse primer for analyzing ClpX gene expression
cttctcagcctctgcttgct

SEQ ID NO: 11 - DNA, 20 bp, Artificial Sequence
  forward primer for analyzing Cp gene expression
tgctctgaacccgagaaagt

SEQ ID NO: 12 - DNA, 20 bp, Artificial Sequence
  reverse primer for analyzing Cp gene expression
ccagagggagcataattcca

SEQ ID NO: 13 - DNA, 20 bp, Artificial Sequence
  forward primer for analyzing beta-actin gene expression
tacaatgagctgcgtgtggc

SEQ ID NO: 14 - DNA, 19 bp, Artificial Sequence
  reverse primer for analyzing beta-actin gene expression
atgtcacgcacgatttccc

SEQ ID NO: 15 - DNA, 20 bp, Artificial Sequence
  forward primer for analyzing GAPDH gene expression
ctgcaccaccaactgcttag

SEQ ID NO: 16 - DNA, 20 bp, Artificial Sequence
  reverse primer for analyzing GAPDH gene expression
gcctctcttgctcagtgtcc